(19) United States
(12) Patent Application Publication
(10) Pub. No.: US 2011/0105889 A1
(43) Pub. Date: May 5, 2011

(54) NANOPARTICLE LABELING AND SYSTEM USING NANOPARTICLE LABELING

(75) Inventors: Kazuya Tsukada, Kanagawa (JP); Natsuki Ito, Tokyo (JP)

(73) Assignee: KONICA MINOLTA MEDICAL & GRAPHIC, INC., Tokyo (JP)

(21) Appl. No.: 13/003,481

(22) PCT Filed: Feb. 20, 2009
(86) PCT No.: PCT/JP2009/053002

§ 371 Date: Jan. 10, 2011

(30) Foreign Application Priority Data
Jul. 17, 2008 (JP) 2008-185857

(51) Int. Cl. A61B 5/055; B32B 1/00; A61B 6/00

(52) U.S. Cl. 600/411; 428/403; 428/404; 600/431

(57) ABSTRACT

A nanoparticle labeling which is simultaneously usable in the combination of X-ray imaging with optical imaging and the combination of X-ray imaging and optical imaging with magnetic resonance imaging characterized by comprising core/shell type semiconductor nanoparticles having an average shell thickness of 0.1 nm or more but not more than 10.0 nm together with an X-ray sensitive material for the former combination, and core/shell type semiconductor nanoparticles having an average shell thickness of 0.1 nm or more but not more than 10.0 nm together with an X-ray sensitive material and magnetic particles for the latter combination.

### TECHNICAL FIELD

The present invention relates to a nanoparticle labeling agent and a system for obtaining an in vivo image with high resolution by using a nanoparticle labeling agent.

### BACKGROUND ART

In the field of clinical image diagnosis, required is a system capable of diagnosing detection of disease and acquisition of positional information of the disease from positional information of a labeling agent in the inside of a body at one time by combining two types of image measuring methods and using various reagents. As a combination of such two different types of modalities, there are a combination of PET and CT, a combination of MRI (magnetic resonance imaging) and an optical imaging, and a combination of an X-ray and an optical imaging.

Since an optical imaging does not expose a patient to ionizing radiation, its degree of acceptance as diagnostic modality is always high. The optical imaging is based on detection of difference in absorption, scattering and/or fluorescence between a normal tissue and a tumor tissue. Fluorescence molecules (namely, an optical imaging agent) emit detectable light rays (that is, light rays with different wavelength) which are discriminable spectrally from an exciting light. The optical imaging agent makes a target/background ratio increase by several digits so that the visibility and distinctiveness of a target region are enhanced.

The optical imaging agent can be designed so as to emit light rays (only under existence of a predetermined enzyme) which are detectable only under the existence of a special event. Such an optical imaging is greatly promised in detecting functional or metabolic changes such as an excessive production of specific protein or enzyme in a body. Further, this is useful, because most diseases induce early functional or metabolic changes in a body before anatomical changes take place. If these metabolic changes can be detected, it becomes possible to detect, diagnose and treat diseases in an early stage, whereby the recovery of patients and/or the chance of curing can be improved.

Although both of the X-ray imaging and the optical imaging provide useful information, both of them do not provide independently all information useful for initial diagnosis of all diseases.

In order to obtain perfect anatomical and functional information, it is known that the utilization of the X-ray imaging together with the optical imaging is beneficial and useful for initial detection of diseases. However, the number of labeling agents capable of being used simultaneously for the X-ray imaging and the optical imaging is few.

As the reason, it is considered that the combination or fusion of particles having different functions makes their original functions lower. Ajiri et al. have reported multifunctional bio imaging nanoparticles GdVO₄:Eu which can be used for magnetic and X-ray imaging in addition to fluorescent imaging (for example, refer to Nonpatent Document 1). However, the above nanoparticles are nanoparticles containing a slight amount of an activator agent, and luminous efficiency is obstructed by surface defects caused at the time that particles are made in nanosize. Further, according to the above report, GdVO₄:Eu nanoparticles with a size controlled to about 30 nm are used. However, with the above size, luminescence intensity becomes low.

Nonpatent Document 1: “Producing quantum dots with nanoengineering and supercritical process” by Masafiumi Ajiri, 15th Bio-imaging Institute Scientific Meeting (public symposium) October, 2006

### DISCLOSURE OF INVENTION

Problems To Be Solved By The Invention

At the present time, a system and method more useful for detecting diseases are required. In addition, a labeling agent capable of being used simultaneously for two different modalities is also required. However, at the present time, only one agent can be used as such a labeling agent capable of being used simultaneously for the X-ray imaging and the optical imaging, and it cannot be said that the agent is enough in respect of sensibility and size. Specifically, in the optical imaging, more high sensibility is required in accordance with the tendency that fluorescent material has more high luminance. Accordingly, the present invention has been achieved to solve these problems, and its object is to provide a labeling agent which can be used simultaneously in the X-ray imaging and the optical imaging.

Further, a magnetic resonance imaging can depict an histological image which cannot be depicted with X-rays. Therefore, if the labeling agent is also used simultaneously in the magnetic resonance imaging, the physical property of substances different from X-rays can be measured. As a result, for example, the labeling agent is useful to improve the judgment of the position and condition of focus sites. Also, it has an advantage which can create new diagnosing technique, such as diagnosis with imaging by multi-modalities.

### MEANS FOR SOLVING THE PROBLEMS

The abovementioned object of the present invention can be attained by the following structures.

1. A nanoparticle labeling agent is characterized by including core/shell type semiconductor nanoparticles with an average shell thickness of 0.1 nm or more and 10.0 nm or less and an X-ray sensitive material.

2. The nanoparticle labeling agent described in the above 1 is characterized in that the X-ray sensitive material contains at least one kind selected from scandium (Sc), titanium (Ti), vanadium (V), chromium (Cr), iron (Fe), cobalt (Co), nickel (Ni), copper (Cu), zinc (Zn), gallium (Ga), selenium (Se), bromine (Br), rubidium (Rb), strontium (Sr), yttrium (Y), zirconium (Zr), niobium (Nb), molybdenum (Mo), ruthenium (Ru), rhodium (Rh), palladium (Pd), silver (Ag), cadmium (Cd), iridium (Ir), tellurium (Te), and iodine (I), caesium (Cs), barium (Ba), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), a tantalum (Ta), tungsten (W), osmium (Os), iridium (Ir), platinum (Pt), and gold (Au).

3. The nanoparticle labeling agent described in the above 1 or 2 is characterized by further including a substance to combine the core/shell type semiconductor nanoparticles and the X-ray sensitive material.

4. The nanoparticle labeling agent described in the above 3 is characterized in that the substance to combine is SiO₂.

5. The nanoparticle labeling agent described in any one of the above 1 to 4 is characterized in that the shell of the core/shell type semiconductor nanoparticles contains SiO₂.

6. The nanoparticle labeling agent described in any one of the above 1 to 5 is characterized by further including magnetic particles.

7. The nanoparticle labeling agent described in the above 6 is characterized in that the magnetic particles are a superparamagnetic substance, a paramagnetic substance, or a ferromagnetic substance.

8. The nanoparticle labeling agent described in the above 7 is characterized in that the superparamagnetic substance, the paramagnetic substance, or the ferromagnetic substance is a metal oxide.

9. The nanoparticle labeling agent described in the above 8 is characterized in that the metal oxide is selected from a group consisting of an oxide of cobalt, an oxide of nickel, an oxide of manganese, and an oxide of iron.

10. The nanoparticle labeling agent described in the above 9 is characterized in that the oxide of iron is Fe₃O₄ or γ-Fe₂O₃.

11. The nanoparticle labeling agent described in the above 7 is characterized in that the paramagnetic substance includes a chelated gadolinium complex as a mother substance and one or more kinds of paramagnetic ions is contained in chelate.

12. The nanoparticle labeling agent described in the above 11 is characterized in that the paramagnetic ions include one or more kinds of manganese (Mn), praseodymium (Pr), neodymium (Nd), samarium (Sm), europium (Eu), terbium (Tb), dysprosium (Dy), holmium (Ho), erbium (Er), thulium (Tm), ytterbium (Yb), and lutetium (Lu).

13. A system for obtaining an in vivo image with high resolution is characterized by comprising a first modality capable of detecting fluorescence of core/shell type semiconductor particles in the nanoparticle labeling agent described in any one of the above 1 to 5, and a second modality capable of detecting absorption of X-ray sensitive material in the nanoparticle labeling agent described in any one of the above 1 to 5, and the system can use the first modality and the second modality simultaneously.

14. The system described in the above 13 for obtaining an in vivo image with high resolution is characterized in that the first modality includes an optical imaging, and the second modality includes an X-ray imaging.

15. A system for obtaining an in vivo image with high resolution is characterized by comprising a first modality capable of detecting fluorescence of core/shell type semiconductor particles in the nanoparticle labeling agent described in any one of the above 6 to 12, a second modality capable of detecting absorption of X-ray sensitive material in the nanoparticle labeling agent described in any one of the above 6 to 12, and a third modality capable of detecting magnetism of magnetic particles in the nanoparticle labeling agent described in any one of the above 6 to 12, and the system can use the first modality, the second modality simultaneously and the third modality simultaneously.

16. The system described in the above 15 for obtaining an in vivo image with high resolution is characterized in that the first modality includes an optical imaging the second modality includes an X-ray imaging and the third modality includes a magnetic resonance imaging.

### EFFECTS OF THE INVENTION

According to the present invention, it becomes possible to provide a nanoparticle labeling agent capable of being used simultaneously in an X-ray imaging and an optical imaging, and in an X-ray imaging, an optical imaging and a magnetic resonance imaging respectively.

### BEST MODE FOR CARRYING OUT THE INVENTION

Hereafter, the present invention is explained in full detail.

### (Core/Shell Type Semiconductor Nanoparticles)

In recent years, in semiconductor ultrafine particles represented by Si, Ge and the like and II-VI group semiconductors such as porous silicon, it is focused that nanostructure crystals of them shows specific optical characteristics is attracts attention. Here, the nanostructure crystal means crystal particles having a particle size with nanometer order of about 1 to 100 nm, and generally it is called with abbreviated names, such as “nanoparticle” and “nanocrystal”.

In the II-VI group semiconductors, when a case where the semiconductors are nanostructure crystals and another case where they are bulk-shaped crystals are compared to each other, they show good optical absorption property and luminescent characteristic in the case where they are the nanostructure crystals. As the reasons, it is considered that in the II-VI group semiconductors being the nanostructure crystals, since quantum size effect exhibits, they have a large band gap as compared with the case of the bulk-shaped crystals. Namely, in the II-VI group semiconductors being the nanostructure crystals, due to the exhibition of quantum size effect, as particle size becomes small, the energy gap of semiconductor nanoparticles becomes large.

In the present invention, the semiconductor nanoparticles have a core/shell structure. In this case, the semiconductor nanoparticles are semiconductor nanoparticles having a core/shell structure constituted with a core particle composed of a semiconductor fine particle and a shell covering the core particle, and it is desirable that the core particle and the shell are different in chemical composition from each other. With this, it is preferable to make the band gap of the shell higher than that of the core.

The shell is required in order to stabilize surface defects of core particles and to raise luminance, and also the shell plays an important role in order to form a surface to which a surface modification agent easily adsorbs and bonds. For the effect of the present invention, this is also an important structure in improving the accuracy of detection sensitivity.

Hereafter, core particles and shell will be explained.

### <Core Particle>

As a semiconductor material used for core particles, various semiconductor materials may be employed. Specific examples of the semiconductor materials include MgS, MgSe, MgTe, CaS, CaSe, CaTe, SrS, SrSe, SrTe, BaS, BaTe, ZnS, ZnSe, ZnTe, CdS, CdSe, CdTe, GaAs, GaP, GaSb, InGaAs, InP, InN, InSb, InAs, AlAs, AlP, AlSb, AlS, PbS, PbSe, Ge, Si, and a mixture of them. In the present invention, an especially preferable semiconductor material is Si.

It is desirable that the average particle size of the core particles relating to the present invention is 0.5 to 15 nm.

In the present invention, the average particle size of the semiconductor nanoparticles is essentially required to be obtained in three dimensions. However, the semiconductor nanoparticles are too fine to be obtained in three dimensions. Therefore, actually, the average particle size is obliged to be determined based on two dimensional images. Accordingly, it is preferable that many electron microscope photographic images of nanoparticles are photographed with different photographing scenes by the use of a transmission electron microscope (TEM) and the average particle size is obtained by the averaging of the photographic images. Herein, the number of nanoparticles to be photographed by TEM is preferably 100 or more.

The average particle size of the semiconductor nanoparticles relating to the present invention is preferably adjusted as the average particle size of core particles so as to emit fluorescence light, namely to emit infrared light in a wavelength range of the infrared region.

Various semiconductor materials may be used as semiconductor materials used for shell. Specific examples of the semiconductor materials include ZnO, ZnS, ZnSe, ZnTe, CdO, CdS, CdSe, CdTe, MgS, MgSe, GaS, GaN, GaP, GaAs, GaSb, InAs, InN, InP, InSb, AlAs, AlN, AlP, AlSb and a mixture of them.

In the present invention, preferable semiconductor materials are SiO₂, GeO₂ and ZnS and SiO₂ is most preferable.

A shell relating to the present invention is not required to cover perfectly on the entire surface of a core, unless the partially exposing portions of the core cause adverse effects.

In the present invention, an average shell thickness is 0.1 nm or more and 10.0 nm or less.

### <Particle Size of a Core/Shell Type Semiconductor Nanoparticle>

The average particle size of the core/shell type semiconductor nanoparticles relating to the present invention is 1 to 10 nm.

It is known that among the core/shell type semiconductor nanoparticleses relating to the present invention, a nanosize particle having a particle size smaller than the wavelength (about 10 nm) of an electron exhibits unique physical properties different from a bulk body, because the influence of size finitude on the movement of an electron becomes large as a quantum size effect.

Generally, a semiconductor nanoparticle, which is a nanometer size semiconductor material and exhibits a quantum confinement effect, is also called “quantum dot”. Such a quantum dot is a small lump in which several hundred to several thousand semiconductor atoms gather, and when a quantum dot becomes an energy exciting state by absorbing light from an excitation source, the quantum dot discharges energy corresponding to an energy band gap of the quantum dot Therefore, if the size or material composition of a quantum dot is adjusted, an energy band gap can be adjusted, whereby energy in wavelength bands at various levels can be utilized. Further, a quantum dot, i.e., a semiconductor nanoparticle has a feature that the adjustment of the size of the nanoparticle with the same composition makes it possible to control its luminous wavelength.

The core/shell type semiconductor nanoparticles relating to the present invention can be adjusted so as to emit light may be emitted fluorescence in the range of 350 to 1100 nm. In the present invention, in order to eliminate the influence of light emission that biological cell itself has and to improve an SN ratio, their light emission with a wavelength of a near infrared region is also used preferably.

### (Production Method of Core/Shell Type Semiconductor Nanoparticles)

As a production method of the core/shell type semiconductor nanoparticles relating to the present invention, production methods with well-known liquid phase method or gas phase method may be used.

Examples of the production methods according to the liquid phase method include a precipitation method, a co-precipitation method, a sol-gel method, a uniform precipitation method, and a reduction method. In addition, a reverse micelle method, a super critical water thermal synthesis method and the like are excellent methods in producing nanoparticles (refer to, for example, Japanese Patent Unexamined Publication Nos. 2002-322468, 2005-239775,10-310770 and 2000-104058).

In the case where semiconductor nanoparticles are produced with the liquid phase process, it is desirable that the production method comprises a process of reducing the precursor of the semiconductor by a reduction reaction. Further, a preferable embodiment comprises a process of conducting a reaction of the precursor in the presence of a surfactant. The semiconductor precursor relating to the present invention is a compound containing elements used as the material of the abovementioned semiconductor. For example, in the case where the semiconductor is Si, examples of the semiconductor precursor include SiCl₄. Other examples of the semiconductor precursor include InCl₃, P(SiMe₃)₃, ZnMe₂, CdMe₂, GeCl₄, tributylphosphine selenium and the like.

The reaction temperature of the semiconductor precursor is not specifically limited, as long as it is equal to or higher than the boiling point of the semiconductor precursor and equal to or lower than the boiling point of a solvent. However, it is preferably in the range of 70 to 110° C.

### (Reducing Agent)

As a reducing agent for reducing the semiconductor precursor, various kinds of known reducing agents may be employed selectively in accordance with reaction conditions. In the present invention, from the viewpoint of the strength of the reducing power, preferred are lithium aluminum hydride (LiAlH₄), sodium boron hydride (NaBH₄), sodium bis(2-methoxyethoxy) aluminum hydride, lithium tri(sec-butyl) boron hydride (LiBH(sec-C₄H₉)₃), potassium tri(sec-butyl) boron hydride and lithium triethyl boron hydride. Especially, from the strength of the reducing power, lithium aluminum hydride (LiAlH₄) is preferred.

### (Solvent)

As a dispersion solvent of the semiconductor precursor, various kinds of known solvents may be employed. However, alcohols such as ethyl alcohol, sec-butyl alcohol and t-butyl alcohol and hydrocarbon solvents such as toluene, decade or hexane is preferably employed. In the present invention, especially, hydrophobic solvents such as toluene and the like are preferred as the dispersion solvent.

### (Surfactant)

As the surfactant, employed may be various kinds of known surfactants which include an anionic surfactant, a nonionic surfactant, a cationic surfactant and an amphoteric surfactant. Among them, preferred are quaternary ammonium salt type surfactants, such as tetrabutylammonium chloride, bromide or hexa fluorophosphates, tetraoctylammonium bromide (TOAB) and tributylhexadecylphosphonium bromide. Especially, tetraoctylammonium bromide is preferred.

The reaction according to the liquid phase method greatly changes depending on the condition of compounds including a solvent in a liquid. In the case where nanosize particles excellent in monodispersity are produced, special attention is required. For example, in a reverse micelle method, since the size and state of reverse micelles becoming a reaction site change depending on the concentration or kind of a surfactant, conditions to form nanoparticles are limited. Accordingly, a proper combination of the surfactant and the solvent is required.

As a production method with a gas phase method, employed are (1) a method of evaporating opposing raw material semiconductors by a first high-temperature plasma generated between electrodes and making them to pass in a second high-temperature plasma generated by electrodeless discharge in a reduced-pressure atmosphere (refer to, for example, Japanese Unexamined Patent Publication No. 6-279015), (2) a method of separating and removing nanoparticles from an anode composed of raw material semiconductors by electrochemically etching (refer to, for example, Japanese Unexamined Patent Publication No. 2003-515459), (3) a laser ablation method (refer to, for example, Japanese Unexamined Patent Publication No. 2004-356163), and (4) a high-speed spattering method (refer to, for example, Japanese Unexamined Patent Publication No. 2004-296781). In addition, a method of making row material gas to cause gas phase reaction on a low pressure condition so as to synthesize a powder containing particles may be also employed preferably.

### <Post Treatment After the Formation of Core/Shell Type Semiconductor Nanoparticles>

In the production method of the core/shell type semiconductor nanoparticles relating to the present invention, a preferable embodiment comprises a process of conducting a post treatment with any one of plasma, heat, radiation, and ultrasonic wave treatment after the formation of semiconductor nanoparticles, especially after the formation of shell.

In the case of plasma treatment, in consideration of particle composition, crystallinity, and surface properties of them, an adaptable treatment, such as low temperature and high temperature plasma, microwave plasma, or atmospheric pressure plasma treatment, may be selected. However, a microwave plasma treatment may be desirable.

With regard to heat treatment, any one of air, vacuum, and inert gas regions is selected, and although heat is applied, an applicable temperature range becomes different depending on the structure of phosphor particles. If temperature is too high, strain may be caused between a core and a shell, or peeling may take place.

In the case of radiation treatment, X-rays, y-rays, and neutron rays which require high energy respectively are used, or vacuum ultraviolet rays (VUV), ultraviolet rays, short pulse laser beams which have low energy respectively are used. The processing time becomes different depending on the type of radiation. Since X-rays and the like have high penetrative power, the radiation treatment is finished iₙ a relatively short time for any kind of composition. In contrast, the radiation treatment with ultraviolet rays needs irradiation for a relatively long time.

Although the principle about the effect of these post treatments has not yet clarified, it is presumed that the post treatments strengthen the jointing power on the interface between a core and a shell of the core/shell type semiconductor nanoparticles and advance passivation, which results in that luminous efficiency is improved. In an infrared luminous body, it is presumed that the effects appear remarkably and reflect on its characteristics.

### (X-ray Sensitive Material)

in the present invention, it is desirable that the X-ray sensitive material contains at least one selected from scandium (Sc), titanium (Ti), vanadium (V), chromium (Cr), iron (Fe), cobalt (Co), nickel (Ni), copper (Cu), zinc (Zn), gallium (Ga), selenium (Se), bromine (Br), rubidium (Rb), strontium (Sr), yttrium (Y), zirconium (Zr), niobium (Nb), molybdenum (Mo), ruthenium (Ru), rhodium (Rh), palladium (Pd), silver (Ag), cadmium (Cd), iridium (Tr), tellurium (re), iodine (I), caesium (Cs), barium (Ba), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf, a tantalum (Ta), tungsten (W), osmium (Os), iridium (Ir), platinum (Pt), and gold (Au).

A material to combine both the core/shell type semiconductor nanoparticles and the X-ray sensitive material becomes different depending on both compositions of the particles. However, the material is to be a compound capable of bonding with both of the compositions. The X-ray sensitive material is metals. Therefore, in consideration of the composition of the core/shell type semiconductor nanoparticles, the compound may be a metal oxide. A preferable example of the compound is silica.

In the present invention, as long as an apparatus can detect fluorescence, the apparatus can be employed as the first modality without specific restriction. For example, a confocal microscope, a two photon microscope, and a microscope for small animals such as OV-100 manufactured by Olympus may be employed. As the second modality, an X-CT capable of measuring X-ray absorption may be employed. As the second modality for small animals, a micro X-CT is suitably employed. As the third modality, a magnetic resonance imaging capable of measuring magnetism may be employed. As the third modality for small animals, a micro MRI is suitably employed.

In the present invention, examples of magnetic particles include a superparamagnetic substance, a paramagnetic substance, and a ferromagnetic substance, and a specific example is a metal oxide. The metal oxide is preferably selected from a group consisting of an oxide of cobalt, an oxide of nickel, an oxide of manganese, and an iron oxide (for example, Fe₃O₄, γ-Fe₂O₃).

The superparamagnetic substance is a substance having magnetism stronger than a ferromagnetic substance, and an example of it includes an iron oxide used as superparamagnetic iron oxide formulation (SPIO). The paramagnetic substance is a substance which has not magnetization when there is no external magnetic field, while when a magnetic field is applied, it shows magnetism magnetized weakly in the direction of the applied magnetic field. Examples of the paramagnetic substance include a crystal containing elements (Fe, Mn, etc.) having an imperfect electron shell, a pyrite, a siderite, and a pyroxene. The ferromagnetic substance is a substance which points out the magnetism of a material in which adjacent spins align in the same direction so that the material has a large magnetic moment as a whole. Accordingly, the ferromagnetic substance can have spontaneous magnetism without being applied with an external magnetic field. There are few elemental substances which show ferromagnetism at a room temperature, and examples of the ferromagnetic substance include iron, cobalt, nickel, and gadolinium.

As the paramagnetic substance, a substance including at least one of a chelated gadolinium complex and a chelate of paramagnetic ion may be employed. As examples of the paramagnetic ion, there are ions including at least one of manganese (Mn), praseodymium (Pr), neodymium (Nd), samarium (Sm), europium (Eu), terbium (Tb), dysprosium (Dy), holmium (Ho), erbium (Er), thulium (Tm), ytterbium (Yb), and lutetium (Lu).

### EXAMPLE

Hereafter, the present invention will be explained with examples. However, the present invention is not limited to these examples.

### Example 1

### <<Production of a Nanoparticle Labeling Agent Containing Core/Shell Type Semiconductor Nanoparticles and X-ray Sensitive Particles>>

### (Preparation of X-ray Sensitive Particles)

In 1000 g of ion exchange water, 1.71 g of barium hydroxide and 0.98 g of sulfuric acid were dissolved respectively, whereby a 0.01 mol/L barium hydroxide solution and a 0.01 mol/L sulfuric acid solution were prepared. Next, the sulfuric acid solution was put into a 2 L flask, stirred at 200 rpm with a paddle made of Teflon (registered trademark), and heated to 100° C., and then, the barium hydroxide solution heated to 100° C. was supplied into the flask over 30 seconds. Thereafter, after the stirring was continued for 3 minutes, the reaction was terminated. Subsequently, the resultant solution was cooled to ordinary temperature and filtered through a filter paper of 5C, and the substance on the filter was washed with ion exchange water, and dried at 105° C. for 3 hours, whereby 2.1 g of powder of barium sulfate was obtained. The particle size of the obtained nanoparticles was 11 nm.

### (Preparation of Fluorescent Si Quantum Dots)

The fluorescent Si quantum dots were prepared as follows.

Into 100 ml of toluene in a flask, 92 μl of SiCl₄ and 1.5 g of tetraoctyl ammonium bromide were added, and stirred at 10000 rpm for 60 minutes by the use of a homogenizer, whereby a reversed micelle was formed. Into the obtained reversed micelle, 2 ml of a 1M-THF solution of LiAIH₄ was added at one time so as to reduce SiCl₄ to Si, and 20 ml of methanol was added to it.

Into the obtained semiconductor nanoparticle solution, 2 ml of 1-heptene and 0.1 ml of a 0.1 M isopropanol solution of H₂PtCl₆ were added, and the added solution was stirred at 10000 rpm for 3 hours. In order to refine the obtained solution, first, toluene and heptene in the abovementioned solution were removed by a rotary evaporator. Subsequently, the refining was conducted in such a way that into the above solution, 100 ml of hexane was added, and further 200 ml of N-methyl formamide was added, and then the resultant solution was shifted to a separating funnel and stirred so as to remove the unreacted reducing agent and surfactant which have shifted into N-methyl formamide. Further, the operations following the addition of 200 ml of N-methyl-formamide was conducted twice, whereby core/shell type semiconductor nanoparticles composed of Si capped by 1-heptene in the hexane were obtained. The obtained nanoparticles had a particle size of 2 nm and a shell thickness of 1 nm.

### (Preparation of a Nanoparticle Labeling Agent Containing X-ray Sensitive Particles and Fluorescent Si Quantum Dots)

Into 100 ml of 2-propanol, 15 mg of the X-ray sensitive particles prepared by the abovementioned method and 15 mg of the Si quantum dots prepared by the above-mentioned method were dispersed for a period of time longer than 30 minutes by ultrasonic treatment.

As a result, these two kinds of particles were fully dispersed into this solution. Then, into the mixture solution, 8.94 ml of 28% ammonia was added as a catalyst, and further 7.5 ml of deionization water was added as a hydrolysis reagent. In an oil bath, this mixture was warmed at a temperature of 40° C. Thereafter, 0.2 ml of tetra ethoxy silan (TEOS) was added into the mixture, and then stirred for 3 hours. These particles were separated by a magnetic concentrator, and washed several times with 2-propanol, deionization water, and alcohol. Further, these particles were subjected to vacuum drying at 110° C. for 6 hours, whereby nanoparticles were obtained. The particle size of the obtained nanoparticles was 19 nm.

### <<Preparation of a Biological Object Labeling Agent>>

Acrylic acid polymer (produced by Wako Pure Chemical Industries, average molecular weight: about 5,000) was dissolved into chloroform, whereby a chloroform solution was prepared. Then, 200 μl of a liquid in which the nanoparticles were dispersed in a chloroform solution, 800 μl of above-mentioned polymer chloroform solution were added into 10 ml of water, and the resultant solution was subjected to ultrasonic irradiation and stirring. Subsequently, chloroform was removed from the solution at 70° C. for 2 hours, whereby a polymer-coated nanoparticle aqueous solution was obtained.

In the case where a biological material is labeled with the above nanoparticles, it is necessary to introduce a functional group which allows both sides of the particles and the biological material to combine with each other. Such an introducing operation was conducted as follows.

### (Introduction of a Surface-Modified Compound to Nanoparticles)

Into the above solution, a buffer salt was added. Further, a surface-modified compound having a polyethylene glycol chain with a molecular weight of 2000 in which an amino group was introduced into one end and a carboxyl group was introduced into one end was selected, and added into the above solution together with carbodiimide as a catalyst, and the resultant solution was stirred at room temperature for 24 hours. In this way, the targeted biological object labeling agent was obtained. By the use of a size selective column which separates selectively respective raw-material components used for the production of the obtained biological object labeling agent and an object and a column which adsorbs chemically, GPC and HPLC treatment were conducted continuously or separately in all columns so as to isolate the biological object labeling agent.

### Example 2

### <<Production of a Nanoparticle Labeling Agent Containing Core/Shell Type Semiconductor Nanoparticles, X-ray Sensitive Particles, and Magnetic Particles>>

### (Preparation of Magnetic Particles)

Into 200 ml of deionization water which was subjected to deoxidization with foams of nitrogen overnight, 1,622 g of iron chloride (III) (FeCl₃) and 5.560 g of iron sulfate (II) (FeSO₄.7H₂O) were dissolved. The concentration ([Fe³⁻]) of the iron (III) ion was 0.05 mol/L, and the concentration ([Fe²⁺]) of the iron (II) ion was 0.10 mol/L. Into the resultant mixture, 1.0 g of polyglycol 4000 was added and dispersed by ultrasonic wave treatment for 30 minutes. This mixture was quickly stirred at a temperature of 60° C. Subsequently, 10 ml of 28% ammonia was quickly added into this mixture. The resultant mixture was quickly stirred for 30 minutes always under the nitrogen atmosphere. Then, superparamagnetic particles were separated by the use of a magnetic concentrator, and washed with deionization water and alcohol several times. Further, these particles were subjected to vacuum drying at a temperature of 60° C. overnight. The dried particles were ground down to nanometer size, whereby deep brown magnetic particles with nanometer size were obtained. The particle size of the obtained magnetic particles was 10 nm.

### (Preparation of X-ray Sensitive Particles)

The X-ray sensitive particles were prepared by the same method as Example 1.

### (Preparation of Fluorescent Si Quantum Dots)

The fluorescent Si quantum dots were prepared by the same method as Example 1.

### (Preparation of Nanoparticles Containing Magnetic Particles, X-ray Sensitive Particles, and Fluorescent Si Quantum Dots)

Into 100 ml of 2-propanol, 15 mg of magnetic particle prepared by the abovementioned method, 15 mg of X-ray sensitive particles prepared by the abovementioned method, and 15 mg of Si quantum dots prepared by the abovementioned method were dispersed by ultrasonic wave treatment for a period of time longer than 30 minutes. As a result, these two kinds of particles were fully dispersed into this solution. Hereafter, nanoparticles were obtained by the use of the same method as Example 1. The obtained nanoparticles had a particle size of 30 nm.

### <<Preparation of a Biological Object Labeling Agent>>

The biological object labeling agent was prepared by the same method as Example 1.

### Comparative Example 1

### <<Production of a Nanoparticle Labeling Agent Containing GdVO₄:Eu Particles and X-ray Sensitive Particles>>

### (Preparation of GdVO₄:Eu Particles)

The GdVO₄:Eu particles were prepared by a supercritical water heat synthesizing method. The obtained particle size was 30 nm.

### (Preparation of X-ray Sensitive Particles)

The X-ray sensitive particles were prepared by the same method as Example 1.

### (Preparation of Nanoparticle Containing GdVO₄:Eu Particles and X-ray Sensitive Particles)

Into 100 ml of 2-propanol, 15 mg of GdVO₄:Eu particles prepared by the abovementioned method and 15 mg of X-ray sensitive particles prepared by the abovementioned method were dispersed by ultrasonic wave treatment for a period of time longer than 30 minutes. As a result, these two kinds of particles were fully dispersed into this solution. Hereafter, nanoparticles were obtained by the use of the same method as Example 1. The obtained nanoparticles had a particle size of 35 nm.

### <<Preparation of a Biological Object Labeling Agent>>

The biological object labeling agent was prepared by the same method as Example 1.

### <<Imaging Evaluation>>

The biological object labeling agents obtained in the above were injected respectively into blood vessels by injection from vein sections of mice. X-ray image contrast, MRI contrast and relative luminescence intensity were measured by the use of Micro X-CT, Micro MRI both manufactured by GE and OV-1000 manufactured by Olympus from images of liver sections in which the biological object labeling agent was collected. The relative luminescence intensity was a luminescence intensity after 2 hour irradiation and was represented on the condition that a luminescence intensity at the time of irradiating initially an exciting light beam for observation was set to 100.

X ray image contrast

AA: extremely clear

A: common as an image and no conspicuous

B: blur with rough sense

MRI contrast

AA: a very good image to allow diagnosis

A: an ordinary common image

B: lack of clearness

TABLE 1

| | | | Relative | |
|---|---|---|---|---|
| | X ray image | | luminescence | |
| | contrast | MRI contrast | intensity | Remarks |
| Example 1 | AA | | 85% | Inventive |
| Example 2 | AA | AA | 80% | Inventive |
| Comparative | B | | 50% | Comparative |
| example 1 | | | | |

In Table 1, it is clearer from the comparison between Example 1 and Comparative example 1 that Example 1 is excellent in any of the X-ray image contrast and the relative luminescence intensity. Further, Example 2 exhibits excellent evaluation results in any of the MRI contrast and the relative luminescence intensity.

1-16. (canceled)

17. A nanoparticle labeling agent, comprising:
core/shell semiconductor nanoparticles each having a core and a shell which covers the core and has an average thickness of 0.1 nm or more and 10.0 nm or less; and
an X-ray sensitive material.

18. The nanoparticle labeling agent of claim 17, wherein the X-ray sensitive material contains at least one X-ray sensitive material selected from scandium (Sc), titanium (Ti), vanadium (V), chromium (Cr), iron (Fe), cobalt (Co), nickel (Ni), copper (Cu), zinc (Zn), gallium (Ga), selenium (Se), bromine (Br), rubidium (Rb), strontium (Sr), yttrium (Y), zirconium (Zr), niobium (Nb), molybdenum (Mo), ruthenium (Ru), rhodium (Rh), palladium (Pd), silver (Ag), cadmium (Cd), iridium (Ir), tellurium (Te), and iodine (I), caesium (Cs), barium (Ba), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), a tantalum (Ta), tungsten (W), osmium (Os), platinum (Pt), and gold (Au).

19. The nanoparticle labeling agent of claim 17, further comprising:
a substance to combine the core/shell type semiconductor nanoparticles and the X-ray sensitive material.

20. The nanoparticle labeling agent of claim 19, wherein the substance to combine is SiO₂.

21. The nanoparticle labeling agent of claim 20, wherein the SiO₂ is contained in the shell of the core/shell type semiconductor nanoparticle.

22. The nanoparticle labeling agent of claim 17, further comprising:
magnetic particles.

23. The nanoparticle labeling agent of claim 22, wherein the magnetic particles are a superparamagnetic substance, a paramagnetic substance, or a ferromagnetic substance.

24. The nanoparticle labeling agent of claim 23, wherein the superparamagnetic substance, the paramagnetic substance, or the ferromagnetic substance is a metal oxide.

25. The nanoparticle labeling agent of claim 24, wherein the metal oxide is selected from a group consisting of an oxide of cobalt, an oxide of nickel, an oxide of manganese, and an oxide of iron.

26. The nanoparticle labeling agent of claim 25, wherein the oxide of iron is Fe₃O₄ or γ-Fe₂O₃.

27. The nanoparticle labeling agent of claim 23, wherein the paramagnetic substance includes a chelated gadolinium complex as a mother substance and one or more kinds of paramagnetic ions contained in a chelating agent.

28. The nanoparticle labeling agent of claim 27, wherein the paramagnetic ions include one or more kinds of manganese (Mn), praseodymium (Pr), neodymium (Nd), samarium (Sm), europium (Eu), terbium (Tb), dysprosium (Dy), holmium (Ho), erbium (Er), thulium (Tm), ytterbium (Yb), and lutetium (Lu).

29. A system for obtaining an in vivo image with high resolution, comprising:
a first modality capable of detecting fluorescence of the core/shell type semiconductor particles in the nanoparticle labeling agent of claim 17, and
a second modality capable of detecting absorption of the X-ray sensitive material in the nanoparticle labeling agent of claim 17,
wherein the first modality and the second modality are adapted to be used simultaneously.

30. The system of claim 29, wherein the first modality includes an optical imaging process, and the second modality includes an X-ray imaging process.

31. A system for obtaining an in vivo image with high resolution, comprising:
a first modality capable of detecting fluorescence of the core/shell type semiconductor particles in the nanoparticle labeling agent of claim 22,
a second modality capable of detecting absorption of the X-ray sensitive material in the nanoparticle labeling agent of claim 22, and
a third modality capable of detecting magnetism of magnetic particles in the nanoparticle labeling agent of claim 22,
wherein the first modality, the second modality and the third modality are adapted to be used simultaneously.

32. The system of claim 31, wherein the first modality includes an optical imaging process, the second modality includes an X-ray imaging process, and the third modality includes a magnetic resonance imaging process.
